(19) United States
(12) Patent Application Publication
(10) Pub. No.: US 2020/0156687 A1
(43) Pub. Date: May 21, 2020

(54) DEVICE FOR DETECTING STATE OF VEHICLE OPERATOR

(71) Applicant: KABUSHIKI KAISHA TOKAI RIKA DENKI SEISAKUSHO, Aichi (JP)

(72) Inventor: Kenji NARUMI, Aichi (JP)

(21) Appl. No.: 16/341,782

(22) PCT Filed: Oct. 5, 2017
(86) PCT No.: PCT/JP2017/036334

§ 371 Date: Apr. 12, 2019

(30) Foreign Application Priority Data
Oct. 19, 2016 (JP) 2016-204884

(51) Int. Cl. B62D 1/04; B60W 40/09; B60W 50/16; G06F 17/14; A61B 5/00

(52) U.S. Cl. CPC B62D 1/046; B60W 40/09; B60W 2040/0818; G06F 17/142; A61B 5/0006; B60W 50/16

(57) ABSTRACT

A device for detecting a state of a vehicle operator includes a first vibration detection unit installed on a vehicle side and configured to detect vibration, a second vibration detection unit installed on a steering part of the vehicle and configured to detect vibration, and a controller configured to process and compute input signals from the first vibration detection unit and the second vibration detection unit. The controller determines a gripping state of the steering part based on a difference between the respective input signals of the first vibration detection unit and the second vibration detection unit.

### CROSS REFERENCE TO RELATED APPLICATIONS

The present application claims the priority of Japanese Patent Application No. 2016-204884, and the entire contents of Japanese Patent Application No. 2016-204884 are incorporated herein by reference.

### TECHNICAL FIELD

The present invention relates to a device for detecting a state of a vehicle operator.

### BACKGROUND ART

A heartbeat fluctuation detection system is known as a device for detecting a state of a vehicle operator according to the prior art (see, for example, Patent Document 1).

The heartbeat fluctuation detection system disclosed in Patent Document 1 includes two electrodes arranged on a steering wheel to detect an electrocardiographic waveform of an operator gripping the steering wheel, a means for analyzing the fluctuation of the heartbeat by performing time domain analysis based on the electrocardiogram waveform of the operator detected through the electrodes, and a means for notifying in accordance with the analysis result.

According to Patent Document 1, the notification processing unit, based on the degree of fluctuation calculated by the heartbeat interval detection processing unit, displays an alarm message on the display/operation unit, produces an alarm sound via the audio output unit, and turns on or blinks the lamp as necessary. This enables the operator to recognize that he/she is feeling sleepy, and operators to be alerted of oncoming vehicles or following vehicles. Further, it is described as being capable of awakening the operator from sleepiness by vibrating the vibration unit as necessary. Therefore, it is described that vehicle accidents are prevented by detecting information of physical condition indicating a state of the operator driving a vehicle and informing the operator.

### CITATION LIST

### Patent Document

Patent Document 1: JP 2008-196194 A

### SUMMARY OF INVENTION

### Technical Problem

Whereas the heartbeat fluctuation detection system disclosed in Patent Document 1 is capable of periodically monitoring the autonomic nervous function of the operator, it has a problem of not being able to accurately determine a temporary syncope or convulsion.

An object of the present invention is to provide a device for detecting a state of a vehicle operator, which can reliably detect the state of an operator (driving posture, drowsiness, incapability to drive).

### Solution to Problem

The present invention provides, as an embodiment, a device for detecting a state of a vehicle operator according to the following [1] to [6].

[1] A device for detecting a state of a vehicle operator, the device including, a first vibration detection unit installed on a vehicle side and configured to detect vibration, a second vibration detection unit installed on a steering part of the vehicle and configured to detect vibration, and a controller configured to process and compute input signals from the first vibration detection unit and the second vibration detection unit, wherein the controller determines a gripping state of the steering part based on a difference between the respective input signals of the first vibration detection unit and the second vibration detection unit.

[2] The device for detecting a state of a vehicle operator according to [1] above, wherein the controller performs Fast Fourier Transform (FFT) on the input signals from the first vibration detection unit and the second vibration detection unit to convert them into signals on a frequency axis, and detects peaks of signals on this frequency axis to determine a state of gripping of the steering part.

[3] The device for detecting a state of a vehicle operator according to [1] or [2] above, wherein the controller determines whether the steering part is in a Free state in which the steering part is not gripped, a Weak state in which the steering part is gently gripped, or a Strong state in which the steering part is strongly gripped.

[4] The device for detecting a state of a vehicle operator according to [3] above, wherein in a case where the difference is greater than a first threshold value, it is determined to be in the Free state, in a case where the difference is less than a second threshold value that is less than the first threshold value, it is determined to be in the Strong state, and in a case where the difference is not greater than the first threshold value and not less than the second threshold value, it is determined to be in the Weak state.

[5] The device for detecting a state of a vehicle operator according to any one of [1] to [4] above, wherein the first vibration detection unit is installed on a steering post configured to support the steering part.

[6] The device for detecting a state of a vehicle operator according to any one of [1] to [5] above, wherein the steering post includes an excitation unit.

### Advantageous Effects of Invention

According to an embodiment of the present invention, it is possible to provide a device for detecting a state of a vehicle operator that can reliably detect the state of the operator (driving posture, drowsiness, incapability to drive).

### BRIEF DESCRIPTION OF DRAWINGS

FIG. 1 is an overall view illustrating an arrangement example of components of a device for detecting a state of a vehicle operator according to an embodiment of the invention.

FIG. 2 is a block diagram illustrating a configuration of the device for detecting a state of a vehicle operator according to the embodiment of the invention.

FIG. 3 illustrates, in the device for detecting a state of a vehicle operator according to the embodiment of the invention, from the top, a vehicle vibration waveform (detection value a1) from the vehicle vibration detection unit, and a steering vibration waveform (detection value a2) from a steering vibration detection unit 20.

FIG. 4 is an explanatory diagram illustrating processing steps from vibration measurement to state determination in the device for detecting a state of a vehicle operator according to the embodiment of the invention.

FIG. 5 is a graph illustrating frequency characteristics indicating each of the amplitudes A on the frequency axis in the vehicle vibration state, the Free state, the Weak state, and the Strong state of the device for detecting a state of a vehicle operator according to the embodiment of the invention.

FIG. 6 is a flowchart illustrating operations of the device for detecting a state of a vehicle operator according to the embodiment of the invention.

### DESCRIPTION OF EMBODIMENT

### First Embodiment of Invention

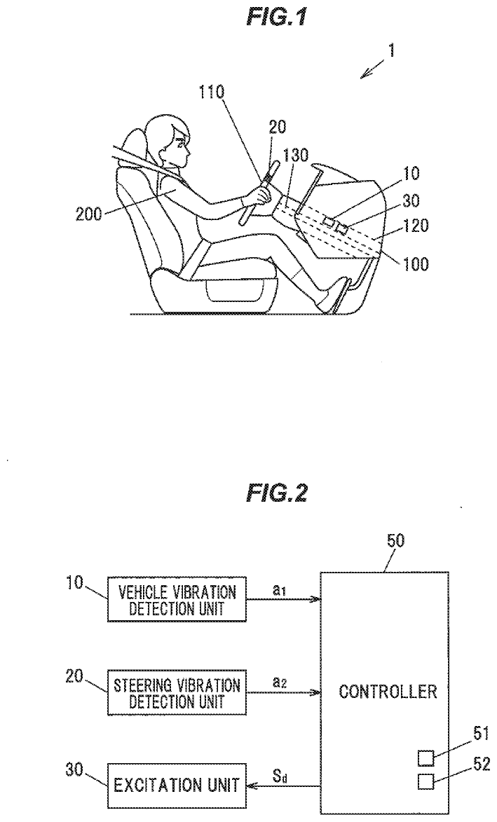

FIG. 1 is an overall configuration diagram illustrating an arrangement example of components of the device for detecting a state of a vehicle operator according to an embodiment of the invention. FIG. 2 is a block configuration diagram illustrating a configuration of the device for detecting a state of a vehicle operator according to the embodiment of the invention. A first embodiment of the invention will be described below as a configuration in which an excitation unit 30 is installed on a steering post 120.

A device for detecting a state of a vehicle operator 1 according to the embodiment of the invention includes, a vehicle vibration detection unit 10, serving as a first vibration detection unit installed on a vehicle 100 as the vehicle side, and configured to detect vibration; a steering vibration detection unit 20, serving as a second vibration detection unit installed on the steering part 110 of the vehicle 100, and configured to detect vibration; and a controller 50 configured to process and compute input signals from the vehicle vibration detection unit 10 and the steering vibration detection unit 20, wherein the controller 50 determines a gripping state of the steering part 110 based on a difference between the respective input signals of the vehicle vibration detection unit 10 and the steering part 110. The vehicle vibration detection unit 10 is installed on a steering post 120 of the vehicle 100, and the excitation unit 30 is installed on the steering post 120.

The device for detecting a state of a vehicle operator 1 according to the embodiment of the invention utilizes the occurrence of natural vibration (resonance) in the steering part 110 and the steering post 120 as the vehicle 100 travels. The amplitude A at the natural frequency f₀ (resonance) varies according to the state in which the operator 200 grips the steering part 110. Namely, detection of the state of the operator 200 is performed by detecting a phenomenon in which when the operator 200 strongly grips the steering part 110, the amplitude A at the natural frequency f₀ (resonance) greatly attenuates, and when the operator 200 does not grip the steering part 110, the attenuation of the amplitude A is small, and the like.

As illustrated in FIG. 1, the steering post 120 is installed on the vehicle 100. The steering post 120 rotatably supports a steering shaft 130. A steering part 110 is installed on an end portion of the steering shaft 130.

A vehicle vibration detection unit 10 is installed on the steering post 120. A steering vibration detection unit 20 is installed on the steering part 110. Further, the excitation unit 30 is installed on the steering post 120. The excitation unit 30 is configured to assist the occurrence of natural vibration (resonance) of the steering part 110 and the steering post 120.

In FIG. 1, during traveling of the vehicle 100, vibration from the engine and the road surface is transmitted from the vehicle 100 to the steering post 120 and the steering part 110. The vehicle vibration detection unit 10 and the steering vibration detection unit 20 detect the strength and the amplitude of this vibration and the vibration of the natural vibration (resonance) generated by this vibration and the vibration generated by the excitation unit 30.

As illustrated in FIG. 2, the vehicle vibration detection unit 10, the steering vibration detection unit 20, and the excitation unit 30 are each connected to the controller 50.

### Vehicle Vibration Detection Unit 10

The vehicle vibration detection unit 10 can use an acceleration sensor. The acceleration sensor is an inertial sensor for measuring acceleration. Acceleration measurement and appropriate signal processing allow various information such as tilt, movement, vibration, and impact to be obtained. While there are many types of acceleration sensors, here, a micro electro mechanical system (MEMS) acceleration sensor in which MEMS technology is applied can be used. The MEMS acceleration sensor includes a detection element portion for detecting acceleration and a signal processing circuit for amplifying and adjusting a signal from a detection element and outputting the resulting signal. For example, an electrostatic capacitance detection type acceleration sensor is a sensor that detects changes in electrostatic capacitance between a moving part and a fixed part of a sensor element.

### Steering Vibration Detection Unit 20

Like the vehicle vibration detection unit 10, the steering vibration detection unit 20 can use an acceleration sensor. The acceleration sensor is an inertial sensor for measuring acceleration. Acceleration measurement and appropriate signal processing allow various information such as tilt, movement, vibration, and impact to be obtained. While there are many types of acceleration sensors, here, a micro electro mechanical system (MEMS) acceleration sensor in which MEMS technology is applied can be used. The MEMS acceleration sensor includes a detection element portion for detecting acceleration and a signal processing circuit for amplifying and adjusting a signal from a detection element and outputting the resulting signal. For example, an electrostatic capacitance detection type acceleration sensor is a sensor that detects changes in electrostatic capacitance between a moving part and a fixed part of a sensor element.

### Excitation Unit 30

The excitation unit 30 is a device that causes intentional vibrations, and the excitation device includes mechanical type, hydraulic type, electrodynamic type, piezoelectric type, and the like. Although various excitation devices can be used, for example, an excitation device using a motor, an excitation device using a magnetostrictive element, or the like can be used. Although the excitation signal generated may be arbitrarily set, in the present embodiment, an impulse signal including a wideband excitation waveform is used. As a result, the natural vibration (resonance) of the steering part 110 and the steering post 120 is excited.

### Controller 50

The controller 50 is, for example, a microcomputer constituted by a Central Processing Unit (CPU) that computes and processes acquired data according to stored programs, a Random Access Memory (RAM) and a Read Only Memory (ROM) which are semiconductor memories, and the like. A program for the operation of the controller 50, a threshold value, and the like, are stored in the ROM, for example. The RAM is used as a storage region that temporarily stores computation results and the like, for example.

The controller 50 includes a determination unit 51 for detecting the state of the operator 200 according to the stored program. Also, A₁ and A₂, which are criteria of state detection as threshold values 52, are stored in the ROM in a referable state as appropriate.

A detection value a1 of vehicle vibration is input from the vehicle vibration detection unit 10 to the controller 50. A detection value a2 of steering vibration is input from the steering vibration detection unit 20 to the controller 50. In addition, the excitation signal Sd is output from the controller 50 to the excitation unit 30.

### Vibration Waveform and Signal Processing

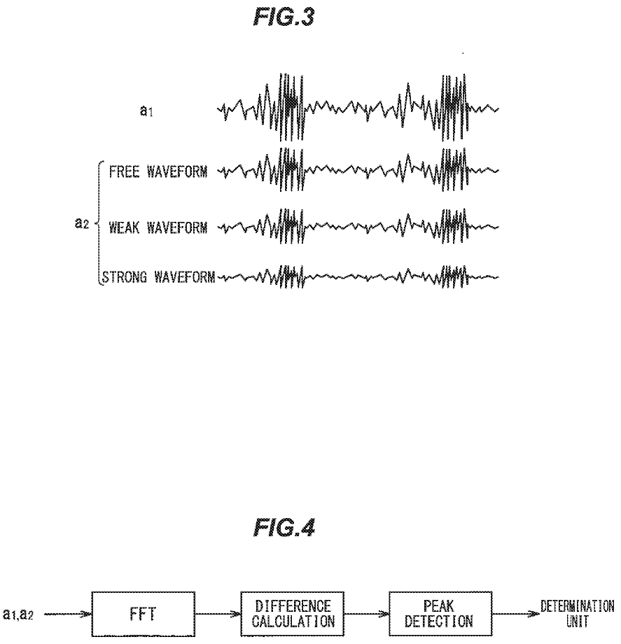

FIG. 3 illustrates, in the device for detecting a state of a vehicle operator according to the embodiment of the invention, from the top, a vehicle vibration waveform (detection value a1) from the vehicle vibration detection unit, and the steering vibration waveform (detection value a2) from the steering vibration detection unit 20. The steering vibration waveform (detection value a2) is a state of gripping of the operator on the steering part 110, namely, a Free waveform for an ungripped Free state, a Weak waveform for a light grip, and a Strong waveform for a strong grip.

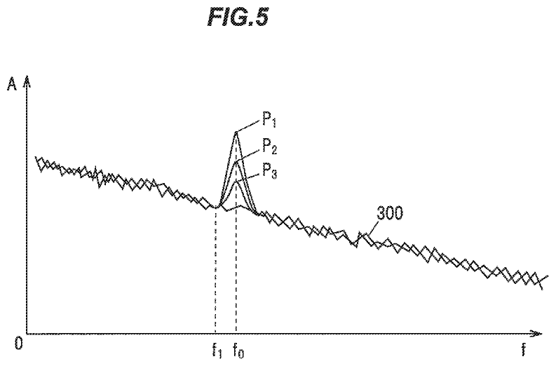

The controller 50 processes the vibration waveform signal illustrated in FIG. 3 in order to determine the gripping state of the steering part 110. FIG. 4 is a diagram illustrating processing steps from vibration measurement to state determination in the device for detecting a state of a vehicle operator according to the embodiment of the invention.

As illustrated in FIG. 4, the vehicle vibration waveform (detection value a1) from the vehicle vibration detection unit 10 and the steering vibration waveform (detection value a2) from the steering vibration detection unit 20 are first converted to signals on the frequency axis by Fast Fourier Transform (FFT). The amplitude A is calculated (vertical axis) as a difference between each detection value a1 and detection value a2. The calculation result of this FFT is obtained as illustrated in FIG. 5 described later. The controller 50 performs a peak detection based on the calculation result, and determines the gripping state of the steering part 110 by the operator based on level determination.

FIG. 5 is a graph illustrating frequency characteristics indicating each of the amplitudes A on the frequency axis in the vehicle vibration state, the Free state, the Weak state, and the Strong state of the device for detecting a state of a vehicle operator according to the embodiment of the invention. The amplitude A is a value based on the difference between the detection value a1 and the detection value a2.

As illustrated in FIG. 5, the natural vibration (resonance) of the steering part 110 and the steering post 120 occurs at the natural frequency f₀. In FIG. 5, a spectrum that illustrates a large amplitude at the natural frequency f₀ is detected in the vibration 300 due to vibration from the engine or the road surface. This is due to the natural vibration (resonance) of the steering part 110 and the steering post 120, and is generated based on the difference between the detection value a1 and the detection value a2.

In FIG. 5, the vibration peak P₁ indicates a spectrum for the Free state where the operator does not grip the steering part 110. In the Weak state where the operator gently grips the steering part 110, the resonance is slightly attenuated to obtain a vibration peak P₂. In the Strong state where the operator grips the steering part 110 strongly, the resonance is further attenuated to obtain a vibration peak P₃.

Note that as illustrated in FIG. 5, since the vibration 300 due to vibration from the engine or the road surface acts as noise, it is preferable that a band below a frequency f₁ lower than the natural frequency f₀ obtained by pre-calculation is cut off by a filter.

Further, since the frequencies of the natural vibrations of the vibration peaks P₁, P₂, and P₃ are slightly shifted by the gripping state of the steering part 110, it is preferable that a level A is detected by means of a peak detection (peak hold).

### Gripping State Determination Operation

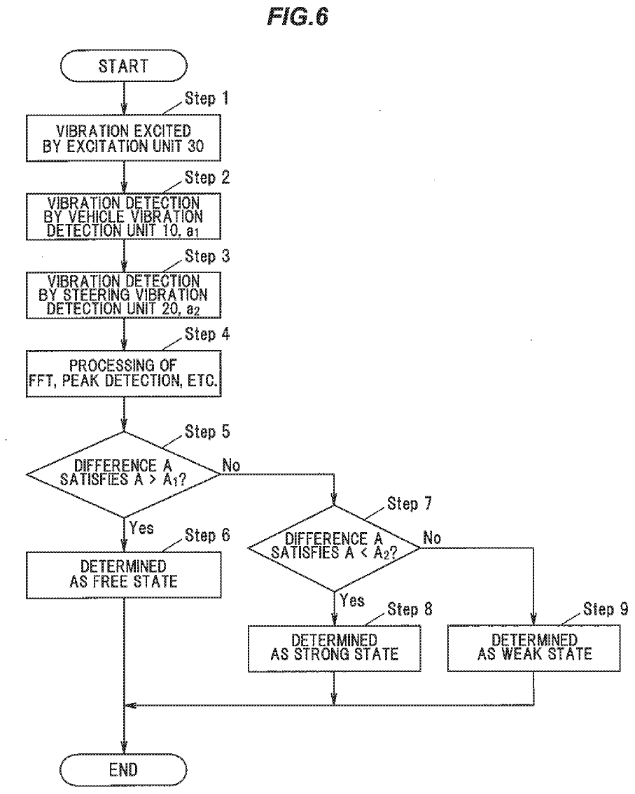

FIG. 6 is a flowchart illustrating operations of the device for detecting a state of a vehicle operator according to an embodiment of the invention. Hereinafter, the operations of the detection of a state of a vehicle operator will be described in accordance with this flowchart.

When the operation of the device for detecting a state of a vehicle operator starts, the excitation unit 30 performs exciting operation (Step 1). The excitation unit 30 applies vibration to the steering part 110 and the steering post 120 by exciting the steering post 120 with the impulse signal. As a result, the natural vibration (resonance) of the steering part 110 and the steering post 120 is excited. Note that the timing of the excitation can be determined to match the timing of signal acquisition by the vehicle vibration detection unit 10 and the steering vibration detection unit 20.

Next, the controller 50 performs vibration detection by obtaining the detection value a1 of vehicle vibration from the vehicle vibration detection unit 10 (Step 2).

In addition, the controller 50 performs vibration detection by obtaining the detection value a2 of vehicle vibration from the steering vibration detection unit 20 (Step 3).

The acquisition of the detection values a1 and a2 of vehicle vibration in Step 2 and Step 3 can be executed in parallel in the case of 2-channel input as illustrated in FIG. 2.

The controller 50 performs signal processing (FFT peak detection) illustrated in FIG. 4 (Step 4).

The controller 50 determines whether the difference A based on the detection value a1 from the vehicle vibration detection unit 10 and the detection value a2 from the steering vibration detection unit 20 satisfies: A>A₁ (Step 5). If A>A₁ is true, the operation proceeds to Step 6 (Step 5: Yes); if A>A₁ is not true, the operation proceeds to Step 7 (Step 5: No).

Note that, A₁ is a threshold value of an amplitude for determining either the Free state or the Weak state. Further, A₂ described later is a threshold value of an amplitude for determining either the Weak state or the Strong state. The amplitude threshold values are set such that A₁>A₂. Namely, the threshold values A₁ and A₂ are set to allow the state to be determined as: the Free state if the amplitude difference A is greater than A₁, the Weak state if the amplitude difference A is in the range of A₁ to A₂, and the Strong state if the amplitude difference A is less than A₂.

The controller 50 can determine that the operator is in a Free state in which the operator does not grip the steering part 110 since the amplitude difference A satisfies: A>A₁ according to the determination unit 51 (Step 6).

The controller 50 determines whether the difference A based on the detection value a1 from the vehicle vibration detection unit 10 and the detection value a2 from the steering vibration detection unit 20 satisfies: A<A₂ (Step 7). If A<A₂ is true, the operation proceeds to Step 8 (Step 7: Yes), and if A<A₂ is not true, the operation proceeds to Step 9 (Step 7: No).

The controller 50 can determine that the operator is in the Strong state in which the operator grips the steering part 110 strongly since the difference A in amplitude satisfies: A<A₂ according to the determination unit 51 (Step 8).

The controller 50 can determine that the operator is in the Weak state in which the operator gently grips the steering part 110 since the difference A in amplitude is not greater than A₁ and not less than A₂ according to the determination unit 51 (Step 9).

The above series of operations can return to Step 1 and can be executed repeatedly. Detection of the state of the operator 200 can be thereby achieved as to whether the operator is in the Free state where the steering part 110 is not gripped, in the Strong state where the steering part 110 is strongly gripped, or in the Weak state where the steering part 110 is gently gripped. Furthermore, reliable detection of the state of the operator (driving posture, drowsiness, incapability to drive) can be achieved based on the detection result of the Free state, Strong state, or Weak state.

### Second Embodiment of Invention

In the configuration illustrated in the first embodiment, the excitation unit 30 is not indispensable as long as the state detection of the vehicle operator is limited to while the vehicle is operated. While the vehicle is traveling, the steering part 110 and the steering post 120 are excited by vibration from the engine and the road surface. As a result, the steering part 110 and the steering post 120 resonate at the natural frequency. Therefore, the peak at the natural frequency f₀ in the frequency characteristic diagram illustrated in FIG. 5 can be detected.

Accordingly, even in the second embodiment configured without the excitation unit 30 illustrated in FIG. 1 and FIG. 2 and omitting the operation of excitation vibration by the excitation unit 30 in Step 1 as described in FIG. 6, it is capable of operating to detect the state of the vehicle operator similarly to the first embodiment.

### Effect of Embodiment of Invention

(1) A device for detecting a state of a vehicle operator 1 according to an embodiment of the invention includes, a vehicle vibration detection unit 10, serving as a first vibration detection unit installed on a vehicle 100 which is on the vehicle side, and configured to detect vibration; a steering vibration detection unit 20, serving as a second vibration detection unit installed on the steering part 110 of the vehicle 100, and configured to detect vibration: and a controller 50 configured to process and compute input signals from the vehicle vibration detection unit 10 and the steering vibration detection unit 20, wherein the controller 50 determines a gripping state of the steering part 110 based on a difference between the respective input signals of the vehicle vibration detection unit 10 and the steering part 110. Detection of the state of the operator 20X) can be thereby achieved as to whether the operator is in the Free state where the steering part 110 is not gripped, in the Strong state where the steering part 110 is strongly gripped, or in the Weak state where the steering part 110 is gently gripped.
(2) Based on the detection results of the Free state, Strong state, and Weak state illustrated above, the controller 50 can presume the state of the operator (driving posture, drowsiness, incapability to drive). It will be possible to apply this as a warning for safe driving and automatically stopping. Since the controller 50 is capable of outputting the detection results of the Free state, Strong state, and Weak state to the in-vehicle device or the like, various presumption, determination, and the like, may be performed on the side of the in-vehicle device based on the detection result of the Free state. Strong state, or Weak state.
(3) In the first embodiment including the excitation unit 30, since the steering part 110 and the steering post 120 are excited by the impulse signal including a wideband excitation waveform, excitation of the natural vibration (resonance) of the steering part 110 and the steering post 120 can be reliably performed.
(4) Even in the configuration of the second embodiment that does not include the excitation unit 30, the peak at the natural frequency f₀ can be detected, since the steering part 110 and the steering post 120 are excited by vibrations from the engine and the road surface. Therefore, operation to detect the state of the vehicle operator can be performed with a simple configuration.

The embodiments of the invention have been described above, however, these embodiments are merely examples and the invention according to claims is not to be limited thereto. These novel embodiments may be implemented in various other forms, and various omissions, substitutions, changes and the like can be made without departing from the spirit and scope of the invention. In addition, all the combinations of the features described in this embodiment are not necessarily essential to solve the problem of the invention. Further, these embodiments are included within the spirit and scope of the invention and also within the invention described in the claims and the scope of equivalents thereof.

### REFERENCE SIGNS LIST

10 Vehicle vibration detection unit
20 Steering vibration detection unit
30 Excitation unit
50 Controller
100 Vehicle
110 Steering part
120 Steering post

1. A device for detecting a state of a vehicle operator, the device comprising:
a first vibration detection unit installed on a vehicle side and configured to detect vibration;
a second vibration detection unit installed on a steering part of the vehicle and configured to detect vibration; and
a controller configured to process and compute input signals from the first vibration detection unit and the second vibration detection unit,
wherein the controller determines a gripping state of the steering part based on a difference between the respective input signals of the first vibration detection unit and the second vibration detection unit.

2. The device for detecting a state of a vehicle operator according to claim 1, wherein the controller performs Fast Fourier Transform (FFT) on the input signals from the first vibration detection unit and the second vibration detection unit to convert them into signals on a frequency axis, and detects peaks of signals on the frequency axis to determine a state of gripping of the steering part.

3. The device for detecting a state of a vehicle operator according to claim 1, wherein the controller determines whether the state of gripping of the steering part is in a Free state in which the steering part is not gripped, in a Weak state in which the steering part is gently gripped, or in a Strong state in which the steering part is strongly gripped.

4. The device for detecting a state of a vehicle operator according to the claim 3, wherein the controller determines the state as the Free state if the difference is more than a first threshold value, as the Strong state if the difference is less than a second threshold value that is less than the first threshold value, and as the Weak state if the difference is not more than the first threshold value and not less than the second threshold value.

5. The device for detecting a state of a vehicle operator according to claim 1, wherein the first vibration detection unit is installed on a steering post that supports the steering part.

6. The device for detecting a state of a vehicle operator according to claim 1, wherein the steering part comprises an excitation unit.
